(19) United States
(12) Patent Application Publication
(10) Pub. No.: US 2025/0368599 A1
(43) Pub. Date: Dec. 4, 2025

(54) PROCESS FOR THE PREPARATION OF CYCLOHOMOGERANATES

(71) Applicants: BASF SE, Ludwigshafen am Rhein (DE); Studiengesellschaft Kohle gGmbH, Mülheim an der Ruhr (DE)

(72) Inventors: Mathias SCHELWIES, Ludwigshafen am Rhein (DE); Bernhard BRUNNER, Ludwigshafen am Rhein (DE); Ralf PELZER, Ludwigshafen am Rhein (DE); Benjamin LIST, Mülheim an der Ruhr (DE); Mathias TURBERG, Mulheim an der Ruhr (DE); Benjamin MITSCHKE, Muelheim an der Ruhr (DE); Sayantani DAS, Muelheim an der Ruhr (DE); Chandra Kanta DE, Muelheim an der Ruhr (DE)

(21) Appl. No.: 18/876,707

(22) PCT Filed: Jun. 21, 2023
(86) PCT No.: PCT/EP2023/066768

§ 371 Date: Dec. 19, 2024

(30) Foreign Application Priority Data
Jun. 22, 2022 (EP) 22180427.1

(51) Int. Cl. C07C 67/303; B01J 27/16

(52) U.S. Cl. CPC C07C 67/303; B01J 27/16; C07C 2601/14; C07C 2601/16

(57) ABSTRACT

The present invention relates to a process for the preparation of cyclohomogeranates. The invention relates to the synthesis of cyclohomogeranates from well available esters of homogeranic acid in only one step. The cyclohomogeranates prepared according to the process of the present invention can be used in the fragrance industry as intermediates or the compounds can be used as such as aroma compound.

### FIELD OF INVENTION

The present invention relates to a process for the preparation of cyclohomogeranates. The invention relates to the synthesis of cyclohomogeranates from well available esters of homogeranic acid in one step.

### BACKGROUND

Cyclohomogeranates are the esters of cyclohomogeranic acid and have been used as synthetic intermediates (Helv. Chem. Acta 1969, 1732-1734). The corresponding esters such as methyl cyclohomogeranate (CAS methyl α-cyclohomogeranate: 64108-19-6; methyl β-cyclohomo-geranate: 2365417-61-2) and ethyl cyclohomogeranate (CAS(S)-ethyl α-cyclohomogeranate: 143658-43-9; ethyl β-cyclohomogeranate: 773136-09-7) have been described several times in the literature (Helv. Chem. Acta 2019, e1900097). Two double bond isomers, the α- and β-isomers, are described.

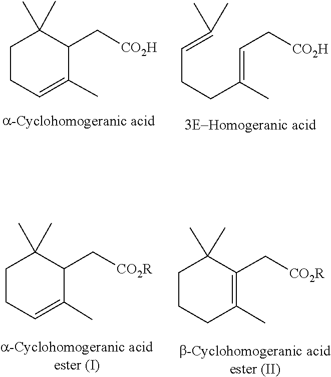

Prior art discloses several processes for the synthesis of cyclohomogeranates which includes processes starting from myrcene, cyclogeranic acid or 2,4,4-trimethyl-2-cyclohexenone. However, no synthetic route has been described starting from precursors such as linalool or homogeranic acid and/or its esters.

Helv. Chem. Acta 2019, e1900097 discloses a synthetic route starting from mycrene. This route involves addition of LiNEt₂ to form the corresponding allylamine (77-87% yield). This step is followed by a cyclization with stoichiometric amounts of H₂SO₄ in 54% yield. The last step is a low yielding (25-31% yield) step, which involves methoxycarbonylation with CO and highly toxic methyl iodide that gives methyl cyclohomogeranate. Thus, the reported overall yield is less than 20% starting from myrcene.

Liebigs Ann. Chem. 1991, 1053-1056 discloses the synthesis of ester of cyclohomogeranic acid starting from 2,4,4-trimethyl-2-cyclohexenone. This route involves the reduction to the alcohol with lithium aluminium hydride, followed by an ortho-ester Claisen rearrangement which results in the formation of ethyl α-cyclohomogeranate.

The route starting from cyclogeranic acid, a compound that is available from geranic acid (Zhurnal Org. Kim. 1991, 27, 2149 and J. für Prakt. Chemie 1936, 147, 199-202) involves seven consecutive steps from cyclogeranic acid to ethyl β-cyclohomogeranate (Helv. Chem. Acta 1969, 1732-1734) resulting in very low overall yields.

Additionally, there are lab-scale processes which cannot be scaled-up to an industrial scale synthesis since reagents produce a lot of waste (for example, silyl protecting groups or MsCl) and also because of limited availability of the starting material. (J. Org. Chem. 1995, 3580-3585 and Angew. Chem. 2000, 569-573).

J. Chem. Soc. Perkin Trans 1, 1983, 1579-1589 discloses the synthesis a positional isomer of methyl cyclohomogeranate, the 2,3-unsaturated methyl cyclohomogeranate (as isomeric E/Z-mixture). The synthetic route starts from 2,4,4-trimethyl-2-cyclohexenone. The synthesis of these 2,3-unsaturated positional isomers is not the purpose of the invention.

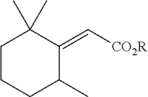

Concluding, the described routes for the synthesis of cyclohomogeranates are either multistep syntheses and low yielding as a consequence or include very low yielding single steps. This makes them not relevant for industrial use. Additionally, many purification steps are involved if the process is a multistep synthesis. This further leads to generation of additional waste and increased cost of energy.

A desired synthesis of α-/β-cyclohomogeranates from homogeranic acid requires the cyclization of an 3,4/7,8 unsaturated ester. Prior art (Liebigs Ann. Chem. 1992, 1049-1053) teaches that for the cyclization step of a different 3,4/7,8 unsaturated ester, stoichiometric amounts of BF₃ are required. Such conditions are not suitable for industrial application since they produce a lot of waste products.

Prior art teaches also that esters of cyclohomogeranic acid lead to the formation of tetrahydroactinidiolide, not the desired cyclohomogeranates (Synthesis 1972, 573-574).

Thus, there is a need to develop an efficient process to synthesize α- or β-cyclohomogeranates or a mixture of both wherein the process is short, scalable, provides good yield and can utilize readily available starting materials.

It is an object of the present invention to provide a process for preparing esters of cyclohomogeranic acid. It is a further object of the invention to provide an economic process for producing esters of cyclohomogeranic acid, which produces α- or β-cyclohomogeranates or a mixture of both in a short sequence of high yielding synthetic steps from well available starting materials. The process should be scalable, should avoid production of waste material and should need only few and simple purification steps.

A further object of the present invention is to arrive at a process which can be run efficiently as a batch or continuous process.

### SUMMARY OF THE INVENTION

We have surprisingly found that α-/β-cyclohomogeranates can be made from technical homogeranic acid in only two synthetic steps. The cyclohomogeranates can be produced as two isomers, the α-cyclohomogeranates or the β-cyclohomogeranates. Also, the α/β-mixture of the isomers can possibly be obtained, and the ratio of the obtained isomeric composition is dependent on the process conditions.

Thus, in one aspect, the present invention relates to the process of synthesizing an ester compound of the general formula (I)

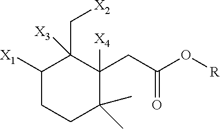

compound of formula (I)
where,
X₁ and X₃ together form a double bond between the carbon atoms to which they are bound, with the proviso that X₂ and X₄ are hydrogen; or
X₃ and X₄ together form a double bond between the carbon atoms to which they are bound, with the proviso that X₁ and X₂ are hydrogen;
or its stereoisomers,
wherein formula (I) comprises,
the compound of formula (Ia)

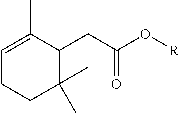

compound of formula (Ia)
or its stereoisomers or mixture of its stereoisomers,
the compound of formula (Ib)

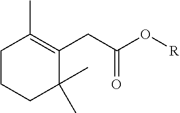

compound of formula (Ib)
wherein R is selected from C₁-C₅ linear or branched alkyl and C₃-C₅ linear or branched alkenyl,
comprising at least the step of:
A) Providing a compound of formula (III)

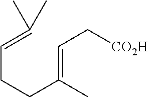

compound of formula (III)
   where R is C₁-C₅ linear or branched alkyl,
   or its stereoisomers, or mixture of its stereoisomers,
B) Cyclizing the compound of formula (III) to obtain compound of formula (I) in the presence of a catalyst selected from Brønsted acid or Lewis acid,
C) Optionally purifying the compound of formula (I) obtained in step B).

In a further aspect, the reaction of the present invention is carried out as a batch process or as a continuous process.

In another aspect, the reaction conditions of the present invention can be varied to obtain a mixture of α-/β-cyclohomogeranate in various ratios.

In a further aspect, the process employed as per the invention results in the formation of compound of formula (IV) or its stereoisomers in an amount of less than 15 wt. %.

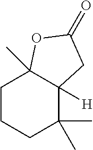

compound of formula (IV)

In another aspect, the process employed as per the invention results in the formation of compound of formula (V) or its stereoisomers

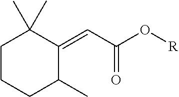

compound of formula (V)
where,
R is selected from C₁-C₅ linear or branched alkyl and C₃-C₅ linear or branched alkenyl, in an amount of less than 10 wt. %.

The compound of formula (V) can be formed in the synthesis of compound (I) or in its purification process by isomerization.

### DETAILED DESCRIPTION OF THE INVENTION

The following detailed description is merely exemplary in nature and is not intended to limit the presently claimed invention or the application and uses of the presently claimed invention. Furthermore, there is no intention to be bound by any theory presented in the preceding technical field, background, summary or the following detailed description.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

Furthermore, the terms “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the subject matter described herein are capable of operation in other sequences than described or illustrated herein. In case the terms “(A)”, “(B)” and “(C)” or AA), BB) and CC) or “(a)”, “(b)”, “(c)”, “(d)”, “(i)”, “(ii)” etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

In the following passages, different aspects of the subject matter are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” or “preferred embodiment” means that a particular feature, structure or characteristic described in connection with the embodiment is included in at least one embodiment of the presently claimed invention. Thus, appearances of the phrases “in one embodiment” or “In a preferred embodiment” or “in a preferred embodiment” in various places throughout this specification are not necessarily all referring to the same embodiment but may refer to the same embodiment. Furthermore, the features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the subject matter, and form different embodiments, as would be understood by those in the art. For example, in the appended claims, any of the claimed embodiments are used in any combination.

Furthermore, the ranges defined throughout the specification include the end values as well, i.e. a range of 1 to 10 implies that both 1 and 10 are included in the range. For the avoidance of doubt, the applicant shall be entitled to any equivalents according to applicable law.

The term C₁-C₅-alkyl denotes a linear or branched alkyl radical comprising 1 to 5 carbon atoms, such as methyl, ethyl, propyl, 1-methylethyl (isopropyl), butyl, 1-methylpropyl, 2 methylpropyl, 1,1-dimethylethyl, pentyl, 1-methylbutyl, 2-methylbutyl, 3-methylbutyl, 2,2-dimethylpropyl, 1-ethylpropyl.

The term “C₃-C₅-alkenyl” refers to a straight-chain or branched unsaturated hydrocarbon radical having 3 to 5 carbon atoms and a double bond in any position.

Examples are “C₃-C₅-alkenyl” groups, such as 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term halogen denotes in each case fluorine, bromine, chlorine or iodine, especially fluorine, chlorine or bromine.

The term Brønsted acid is used herein as defined by IUPAC for a molecular entity (atom, ion, molecule, compound, complex, etc.), that is capable of donating one or more protons to another chemical species.

The term Lewis acid is used herein as defined by IUPAC for a molecular entity that is an electron-pair acceptor and therefore able to react with a Lewis base to form a Lewis adduct, by sharing the electron pair furnished by the Lewis base.

The terms “cyclohomogeranate” and “ester of cyclohomogeranic acid” are used interchangeably in the present specification. The term cyclohomogeranate includes the α- or the β-isomer, γ-isomer or the mixtures of both the isomers unless specified. Accordingly, for example the terms Methyl α-cyclohomogerante and α-cyclohomogeranic acid methylester are used interchangeably.

As used herein the terms “compound (X) or its stereoisomers or mixture of its stereoisomers” refers to the compound(s) of formula (X) including all stereoisomeric forms (stereoisomers) thereof in all ratios. Thus, the term “compound of formula (Ia) or its stereoisomers or mixture of its stereoisomers” refers to the compound Ia in its racemic form, or to one of its enantiomerically pure forms (R or S), or to a mixture of the two possible enantiomers in any ratio, where the ratio of the enantiomers is in the range of 0.01:99.99 to 99.99 to 0.01.

The term “stereoisomer” is a general term as described by IUPAC that is used for all isomers of individual compounds that differ only in the arrangement of their atoms in space, not in the connectivity of the atoms. Thus, the term stereoisomer includes mirror image isomers (enantiomers), geometric (cis/trans or E/Z) isomers, and diastereoisomers. For precise definitions of the terms, see the IUPAC definition or G. Helmchen: “Vocabulary and Nomenclature of Organic Stereochemistry”. In Houben-Weyl E21a, Stereoselective Synthesis. Helmchen, R. W. Hoffmann, J. Mulzer, E. Schaumann (Hrsg.), 1995, 1-74. The possible isomers can be present as mixtures (i.e. racemates, cis/trans-mixtures or mixtures of diasteroisomers).

The presently claimed invention relates to a process for preparing an ester compound of the general formula (I)

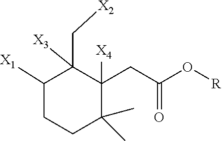

compound of formula (I)
where,
X₁ and X₃ together are the second bond of a double bond between the carbon atoms to which they are bound, with the proviso that X₂ and X₄ are hydrogen; or
X₃ and X₄ together are the second bond of a double bond between the carbon atoms to which they are bound, with the proviso that X₁ and X₂ are hydrogen;
or its stereoisomers,
wherein formula (I) comprises,
the compound of formula (Ia)

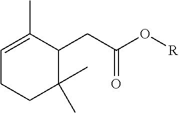

compound of formula (Ia)
or its stereoisomers or mixture of its stereoisomers,
and the compound of formula (Ib)

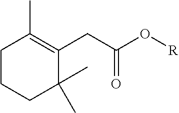

compound of formula (Ib)
wherein R is selected from C₁-C₅ linear or branched alkyl and C₃-C₅ linear or branched alkenyl,
comprising at least the step of:
A) Providing a compound of formula (III)

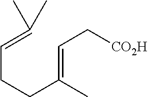

compound of formula (III)
where R is C₁-C₅ linear or branched alkyl,
or its stereoisomers, or mixture of its of stereoisomers,
B) Cyclizing the compound of formula (III) to obtain compound of formula (I) in the presence of a catalyst selected from Brønsted acid or Lewis acid,
C) Optionally purifying the compound of formula (I) obtained in step B).

In an embodiment, R is selected from methyl, ethyl, propyl, butyl, isobutyl, isopropyl, 1-propenyl, or 2-propenyl.

Preferably R is selected from methyl or ethyl.

### Catalyst

In an embodiment, the catalyst in step B) is selected from the group consisting of Lewis acid or Brønsted acid.

In another embodiment, the catalyst in step B) is selected from the group consisting of
Lewis acids in the form of MAₓ where M is a metal, and A is a non-coordinating, weakly coordinating anion or a halogen and x is the valence of M.
   Or
Brønsted acid selected from the group consisting of mineral acids, mineral acid salts, organic acids, acid anhydrides (that can act as Brønsted acid precursors and can form free acids upon contact with a protic reagent), solid acid catalyst, or combinations thereof.

In an embodiment, the catalyst in step B) is Lewis acid in the form of MAₓ where M is a metal, and A is a non-coordinating, weakly coordinating anion, alkoholate or a halogen and x is the valence of M wherein M comprises a transition metal, lanthanoid metal, or metals from Group 2, 3, 4, 5, 12, 13, 14 and 15 of the periodic table of the elements, and combinations thereof.

The Lewis acid (also referred to as the Lewis acid catalyst) may be any Lewis acid based on transition metals, lathanoid metals, and metals from Group 2, 3, 4, 5, 12, 13, 14 and 15 of the periodic table of the elements.

In an embodiment, the metal M is selected from the group of elements iron, magnesium, zinc, boron, scandium, yttrium, lanthanum, europium, zirconium, titanium, manganese, aluminium, ytterbium, tin, vanadium, bismuth, scandium, or hafnium.

The catalysts of the present invention are Lewis acids, such as a metal salt catalyst of general formula MAₓ wherein A is a non-coordinating or weakly coordinating anion and M is a Group IIIB, rare earth or lanthanide, actinide or Group IVB cation with x being the valence of M. By the term “non-coordinating or weakly coordinating anion” it is meant that the anion is not bound to the metal in an aqueous solution. Examples of a non-coordinating or weakly coordinating anion in the present inventions are trifluoromethane sulfonate, also known as triflate ([CF₃SO₃]⁻), hexafluorophosphate ([PF₆]⁻), [Al[OC(CF₃)₃]₄]⁻, tetrafluoroborate ([BF₄]⁻), perchlorate ([ClO₄]⁻), teflate ([TeOF₅]⁻), BArF ([B(ArHₓF_{y})₄]⁻ where Ar is an aryl and x+y=5, e.g., [B(C₆F₅)₄]⁻, tosylate ([CH₃C₆H₄SO₃]⁻, mesylate ([CH₃SO₃]⁻) and antimonyhexafluoride ([SbF₆]⁻).

It should be noted that whether a particular anion is “non-coordinating or weakly coordinating” is dependent on its environment, e.g., solvent, presence of impurities and, especially, the cation.

Examples of Group IIIB metals are scandium and yttrium. An example of Group IVB metal is hafnium. Examples of rare earth or lanthanide cation are lanthanum, europium and ytterbium. Examples of water tolerant Lewis acids in the present invention are scandium triflate [Sc(CF₃SO₃)₃], europium triflate [Eu(CF₃SO₃)₃], hafnium triflate [Hf(CF₃SO₃)₄], yttrium triflate [Y(CF₃SO₃)₃], lanthanum triflate [La(CF₃SO₃)₃] and ytterbium triflate [Yb(CF₃SO₃)₃]. Many of these water tolerant Lewis acids are commercially available or can be synthesized by methods known in the art.

In a preferred embodiment, the Lewis acid is selected from scandium triflate [Sc(CF₃SO₃)₃], aluminium triflate [Al(CF₃SO₃)₃], hafnium triflate [Hf(CF₃SO₃)₄], yttrium triflate [Y(CF₃SO₃)₃], bismuth triflate [Bi(CF₃SO₃)₃] or ytterbium triflate [Yb(CF₃SO₃)₃],

Lewis acid based on transition metals, lathanoid metals, and metals from Group 2, 3, 4, 5, 12, 13, 14 and 15 generally are designated by the formula MX₄; wherein M is a transition metal or a Group 2, 4, 5, 12, 13, or 14 metal, and X is a halogen independently selected from the group consisting of fluorine, chlorine, bromine, and iodine. X may also be a pseudohalogen. Examples include titanium tetrachloride, titanium tetrabromide, vanadium tetrachloride, tin tetrachloride and zirconium tetrachloride.

The Group 4, 5, or 14 Lewis acids may also contain more than one type of halogen. Examples include titanium bromide trichloride, titanium dibromide dichloride, vanadium bromide trichloride, and tin chloride trifluoride.

In an embodiment, A is a halogen selected from the group of chlorine, fluorine and bromine, preferably chlorine.

In a preferred embodiment, the Lewis acid is selected from FeCl₃, FeBr₃, Me₂AlCl, TiCl₃(OiPr), AlCl₃, ZnCl₂, MnCl₂, MgCl₂, MnCl₂, BCl₃, BiCl₃, SbCl₅ and its salts, SiCl₄, InCl₃ and its salts, GaCl₃, ZrCl₄, NbCl₅, TaCl₅, and its salts, BF₃, SnCl₄ and TiCl₄; more preferably FeCl₃.

In a preferred embodiment, the Lewis acid is selected from scandium triflate [Sc(CF₃SO₃)₃], aluminium triflate [Al(CF₃SO₃)₃], hafnium triflate [Hf(CF₃SO₃)₄], yttrium triflate [Y(CF₃SO₃)₃], bismuth triflate [Bi(CF₃SO₃)₃] or ytterbium triflate [Yb(CF₃SO₃)₃], FeCl₃, FeBr₃, Me₂AlCl, TiCl₃(OiPr), AlCl₃, ZnCl₂, MgCl₂, BCl₃, Al(OTf)₃, BF₃, SnCl₄, or TiCl₄.

Group 4, 5 and 14 Lewis acids useful in the method may also have the general formula MRₙX₄₋ₙ; wherein M is Group 4, 5, or 14 metal; wherein R is a monovalent hydrocarbon radical selected from the group consisting of C-1 to C-12 alkyl, aryl, arylalkyl, alkylaryl and cycloalkyl radicals; wherein n is an integer from 0 to 4; and wherein X is a halogen independently selected from the group consisting of fluorine, chlorine, bromine, and iodine, preferably chlorine. X may also be a pseudohalogen. Examples include benzyltitanium trichloride, dibenzyltitanium dichloride, benzylzirconium trichloride, dibenzylzirconium dibromide, methyltitanium trichloride, dimethyltitanium difluoride, dimethyltin dichloride and phenylvanadium trichloride.

Group 4, 5 and 14 Lewis acids useful in method may also have the general formula M(RO)ₙR′ₘX₍ₘ₊ₙ₎; wherein M is Group 4, 5, or 14 metal; RO is a monovalent hydrocarboxy radical selected from the group consisting of C1 to C30 alkoxy, aryloxy, arylalkoxy, alkylaryloxy radicals; R′ is a monovalent hydrocarbon radical selected from the group consisting of C1 to C12 alkyl, aryl, arylalkyl, alkylaryl and cycloalkyl radicals; n is an integer from 0 to 4; m is an integer from 0 to 4 such that the sum of n and m is 3 or 4; and X is a halogen independently selected from the group consisting of fluorine, chlorine, bromine, and iodine, preferably chlorine. X may also be a psuedohalogen. Examples include methoxytitanium trichloride, n-butoxytitanium trichloride, di (isopropoxy) titanium dichloride, phenoxytitanium tribromide, phenylmethoxyzirconium trifluoride, methyl methoxytitanium dichloride, methyl methoxytin dichloride and benzyl isopropoxyvanadium dichloride.

Group 5 Lewis acids may also have the general formula MOX₃; wherein M is a Group 5 metal; X is a halogen independently selected from the group consisting of fluorine, chlorine, bromine, and iodine, preferably chlorine. An example is vanadium oxytrichloride.

The Group 13 Lewis acids useful in method may also have the general formula: MRₙX₃₋ₙ wherein M is a Group 13 metal; R is a monovalent hydrocarbon radical selected from the group consisting of C1 to C12 alkyl, aryl, arylalkyl, alkylaryl and cycloalkyl radicals; and n is a number from 0 to 3; and X is a halogen independently selected from the group consisting of fluorine, chlorine, bromine, and iodine, preferably chlorine. X may also be a pseudohalogen. Examples include ethylaluminum dichloride, methylaluminum dichloride, benzylaluminum dichloride, isobutylgallium dichloride, diethylaluminum chloride, dimethylaluminum chloride, ethylaluminum sesquichloride, methylaluminum sesquichloride, trimethylaluminum and triethylaluminum.

Group 13 Lewis acids useful in this disclosure may also have the general formula M(RO)ₙR′ₘX₃₋₍ₘ₊ₙ₎; wherein M is a Group 13 metal; RO is a monovalent hydrocarboxy radical selected from the group consisting of C1 to C30 alkoxy, aryloxy, arylalkoxy, alkylaryloxy radicals; R′ is a monovalent hydrocarbon radical selected from the group consisting of C-1 to C-12 alkyl, aryl, arylalkyl, alkylaryl and cycloalkyl radicals; n is a number from 0 to 3; m is an number from 0 to 3 such that the sum of n and m is not more than 3; and X is a halogen independently selected from the group consisting of fluorine, chlorine, bromine, and iodine, preferably chlorine. X may also be a psuedohalogen. Examples include methoxyaluminum dichloride, ethoxyaluminum dichloride, 2,6-di-tert-butylphenoxyaluminum dichloride, methoxy methylaluminum chloride, 2,6-di-tert-butylphenoxy methylaluminum chloride, isopropoxygallium dichloride and phenoxy methylindium fluoride.

Group 13 Lewis acids useful in this disclosure may also have the general formula M(RC(O)O)ₙR′ₘX₃₋₍ₘ₊ₙ₎; wherein M is a Group 13 metal; RC (O) O is a monovalent hydrocarbacyl radical selected from the group consisting of C2 to C30 alkacyloxy, arylacyloxy, arylalkylacyloxy, alkylarylacyloxy radicals; R′ is a monovalent hydrocarbon radical selected from the group consisting of C1 to C12 alkyl, aryl, arylalkyl, alkylaryl and cycloalkyl radicals; n is a number from 0 to 3 and m is a number from 0 to 3 such that the sum of n and m is not more than 3; and X is a halogen independently selected from the group consisting of fluorine, chlorine, bromine, and iodine, preferably chlorine. X may also be a pseudohalogen. Examples include acetoxyaluminum dichloride, benzoyloxyaluminum dibromide, benzoyloxygallium difluoride, methyl acetoxyaluminum chloride, and isopropoyloxyindium trichloride.

In an embodiment, in the presence of water, some Lewis acids may decompose to from Brønsted acids.

The term Brønsted acid is used herein as defined by IUPAC for a molecular entity (atom, ion, molecule, compound, complex, etc.), that is capable of donating one or more protons to another chemical species.

In an embodiment, the catalyst in step B) is a Brønsted acid selected from the group consisting of mineral acids, mineral acid salts, organic acids, acid anhydrides (that can act as Brønsted acid precursors and can form free acids upon contact with a protic reagent), solid acid catalyst, zeolites, acidic ion exchange resins and combinations thereof.

In an embodiment, the Brønsted acid selected from the group consisting of mineral acids, mineral acid salts, organic acids, solid acid catalyst, or combinations thereof.

In an embodiment, the mineral acids selected from hydrochloric acid, nitric acid, phosphoric acid, sulfuric acid, boric acid, hydrofluoric acid, hydrobromic acid, hydroiodic acid, or phosphonic acid.

In an embodiment, the mineral acids are immobilized on silica or any other thermostable support.

In another embodiment, the mineral acid salts selected from potassium bisulfate, sodium bisulfate, sodium dihydrogen phosphate.

In a further embodiment, the organic acids selected from p-toluenesulfonic acid, methansulfonic acid, formic acid, acetic acid, oxalic acid, or trifluoroacetic acid.

In another embodiment, an acid anhydride is used as catalyst. Acid anhydrides act as acid precursors and can form Brønsted acids upon contact with a protic reagent. In an embodiment, the acid anhydride is selected from phosphorous pentoxide (P₂O₅), carbon dioxide (CO₂), sulphur trioxide (SO₃), acetic anhydride (Ac₂O), methanesulfonic acid anhydride.

In another embodiment, the solid acid catalyst can be used independently or alternatively can be utilized in combination with one or more mineral acid or other types of catalysts. Exemplary solid acid catalysts which can be utilized include, heteropolyacids, acid resin-type catalysts, mesoporous silicas, acid clays, sulfated zirconia, molecular sieve materials, zeolites, and acidic material on a thermostable support. Where an acidic material is provided on a thermostable support, the thermostable support can include for example, one or more of silica, tin oxide, zirconia, titania, carbon, alpha-alumina, and the like. The oxides themselves (e.g., ZrO₂, SnO₂, TiO₂, etc.) which may optionally be doped with additional acid groups such as SO₄²⁻ or SO₃H⁻ may also be used as solid acid catalysts.

The terms “solid acid” and “solid acid catalyst” are used synonymously herein and can comprise one or more solid acid materials.

Further examples of solid acid catalysts include strongly acidic ion exchangers such as cross-linked polystyrene containing sulfonic acid groups. For example, the Amberlyst®-resins are functionalized styrene-divinylbenzene copolymers with different surface properties and porosities. The functional group is generally of the sulfonic acid type. The Amberlyst®-brand resins are supplied as beads (Amberlyst® is a registered trademark of the Dow Chemical Co.). Similarly, Nafion®-brand resins are sulfonated tetrafluoroethylene-based fluoropolymer-copolymers which are solid acid catalysts. Nafion® is a registered trademark of E.I. du Pont de Nemours & Co.), DOWEX 50WX8® is an ion exchange resin with styrene-divinylbenzene copolymer matrix with sulfonic acid functional groups. (It is a registered trademark of The Dow Chemical company).

Solid catalysts can be in any shape or form now known or developed in the future, such as granules, powder, beads, pills, pellets, flakes, cylinders, spheres, or other shapes.

Supports for metal catalysts can be any suitable support (now known or developed in the future) that is sufficiently robust to withstand the reaction conditions disclosed herein. Suitable catalyst supports include alumina, carbon, ceria, magnesia, silica, titania, zirconia, zeolites (preferably, Y, ZSM 5, MWW and beta), hydrotalcite, molecular sieves, clays, iron oxide, silicon carbide, aluminosilicates, and modifications, mixtures or combinations thereof.

Zeolites may also be used as solid acid catalysts. Of these, H-type zeolites are generally preferred, for example zeolites in the mordenite group or fine-pored zeolites such as zeolites X, Y and L, e.g., mordenite, erionite, chabazite, or faujasite. Also suitable are ultrastable zeolites in the faujasite group which have been dealuminated.

In a preferred embodiment, the step B) is carried out in the presence of an Brønsted acid selected from phosphoric acid, p-toluenesulfonic acid, phosphonic acid or strongly acidic ion exchangers.

Preferably the Brønsted acid is selected from phosphoric acid or trifluoracetic acid. More preferably phosphoric acid.

In an embodiment, the mineral acid specifically phosphoric acid is immobilized on silica or any other thermostable support.

In a preferred embodiment, the phosphoric acid is an aqueous solution, which is 50% aqueous solution, 80% aqueous solution or 85% aqueous solution.

In another preferred embodiment, the phosphoric acid used is in its crystalline form.

In another preferred embodiment, polyphosphoric acid is used as catalyst.

In a preferred embodiment of the present invention the catalyst of step B) is selected from FeCl₃, scandium triflate [Sc(CF₃SO₃)₃], aluminium triflate [Al(CF₃SO₃)₃], hafnium triflate [Hf(CF₃SO₃)₄], yttrium triflate [Y(CF₃SO₃)₃], Bismuth triflate [Bi(CF₃SO₃)₃], ytterbium triflate [Yb(CF₃SO₃)₃], phosphoric acid (85% aqueous solution), phosphoric acid (crystalline) or polyphosphoric acid.

Depending on the type of catalyst the ratio of the alpha and the beta isomer in the final product varies. Thus, depending on the requirement of the final product the reaction conditions, specifically the choice of catalyst can be varied to obtain the alpha and beta isomers in varied proportions. The choice of the catalyst also influences the formation of the side product.

Thus, the judicious choice of either suitable Brønsted or Lewis acid catalysts enables to alter the isomeric ratio in favor of either α- or β-isomer of the cyclohomogeranates.

In an embodiment, the catalyst in the reaction is present in an amount in the range of 0.01 to 100 mol % based on total amount of compound of formula (III).

In another embodiment, the catalyst in the reaction is present in an amount in the range of 1 to 50 mol % based on total amount of compound of formula (III).

In a preferred embodiment, catalyst in the reaction is present in an amount in the range of 2.5 mol % to 25 mol % based on total amount of compound of formula (III), more preferably in the range of 5 mol % to 25 mol % based on total amount of compound of formula (III), even more preferably in the range of 5 mol % to 10 mol % based on total amount of compound of formula (III).

In a preferred embodiment, catalyst in the reaction is present in an amount in the range of 2.5 mol % to 25 mol % based on total amount of compound of formula (III), more preferably in the range of 5 mol % to 25 mol % based on total amount of compound of formula (III), even more preferably in the range of 5 mol % to 10 mol % based on total amount of compound of formula (III), wherein the catalyst is crystalline phosphoric acid.

In a preferred embodiment, the temperature in step B) is in the range of 0° C. to 150° C., in particular the temperature is in the range of 20° C. to 120° C., preferably in the range of 50° C. to 120° C.

In a more preferred embodiment, the temperature in step B) is at every temperature in between 80° C. and 120° C.

In a further embodiment, step B) is carried in the presence or absence of solvent.

In an embodiment, the solvent is selected from the group consisting of ketones, esters, aromatic solvents, aliphatic solvents, cyclic ethers, alcohols, water, nitriles, ethers and mixtures thereof.

In another embodiment, the solvent is selected from toluene, benzene, benzyl alcohol, chlorobenzene, benzonitrile, xylene, trifluorotoluene, nitrobenzene, cyclohexane, or n-heptane, hexane, octane, tetrahydrofuran, 2-methyltetrahydrofuran, methyl-tert-butyl ether, 1-pentanol, 1-hexanol, methanol, 1-butanol, 1-propanol, 2-propanol, acetonitrile, water, dimethylformamide, tetrahydrofuran, toluene, ethyl acetate, dichloromethane, 1,1,1,3,3,3-hexafluoroisopropanol, dioxane or ethanol.

Preferably the solvent is selected from toluene, cyclohexane, n-heptane, ethanol or methanol.

The process in step B) may be performed as a batch or semi-continuous or a continuous process on an industrial scale. The choice of the optimal setup is dependent on many factors such as the phase behavior of the reaction system (biphasic liquid/liquid system or reaction in one homogeneous phase with a dissolved acid catalyst or liquid phase with a solid catalyst and) the required stirring, the production volume, the required reaction temperature, the necessary residence times and many others.

In an embodiment, the reaction is carried out as a batch reaction for a time period in the range of 10 minutes to 24 hours, preferably for a time period in the range of 10 min to 10 hours, more preferably for a time period in the range of 10 min to 5 hours.

In another embodiment, the reaction is carried out in a continuous reactor setup such as a mixing pump with a residence time in the range of 1 min to 10 hours, preferably for a time period in the range of 1 min to 5 hours, more preferably for a time period in the range of 1 min to 2 hours.

In an embodiment, the compounds of formula (I) are selected from

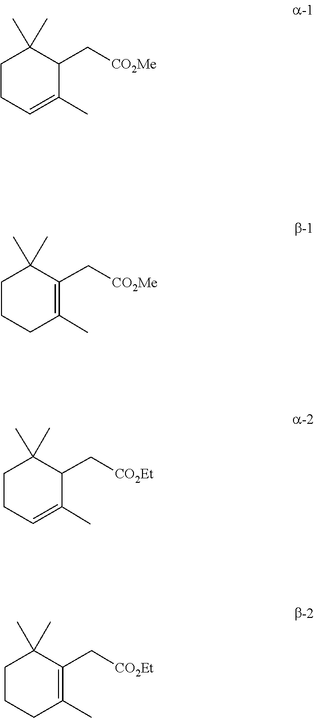

In an embodiment, the compound of formula (I) includes a compound of formula (Ic)

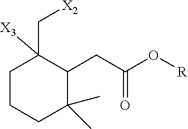

Compound of formula (Ic)
where,
X₂ and X₃ together form a double bond between the carbon atoms to which they are bound,
R is selected from C₁-C₅ linear or branched alkyl and C₃-C₅ linear or branched alkenyl.

In an embodiment, the compound of formula (I) includes a compound of formula (Ic), wherein compound of formula (Ic) is γ-1 or γ-2

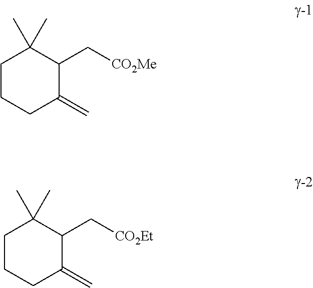

In an embodiment, the compound of formula (I) includes a compound of formula (IV) in an amount of less than 15%, preferably less than 10%, more preferably less than 5%.

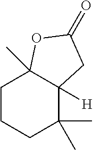

compound of formula (IV)

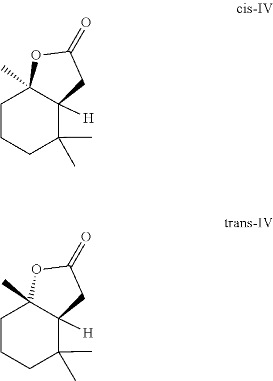

Compound of formula (IV) (tetrahydroactinidiolide), in particular cis-IV may be formed as side product in the reaction. The formation of this side product is dependent on the process conditions. However, by modifying the process conditions the amount of this side product can be controlled.

In an embodiment, the compound of formula (I) includes compound of formula (V)

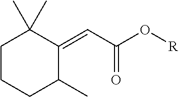

compound of formula (V)
or its stereoisomers or mixture of its stereoisomers,
where,
R is selected from C₁-C₅ linear or branched alkyl and C₃-C₅ linear or branched alkenyl.
   in an amount of less than 10 wt. %, preferably less than 9 wt. %, more preferably less than 8 wt. %.

The compound of formula (V) can be formed in the synthesis of compound (I) or in its purification process by isomerization.

In an embodiment, the compound of formula (III) is obtained by a process comprising at least the steps of:
AA) providing a compound of formula (II)

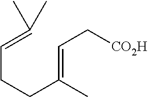

compound of formula (II)
BB) Subjecting the compound of formula (II) to esterification reaction to obtain a compound of formula (III).

The compound of formula (II), Homogeranic acid (mix of isomers) can be converted into the respective homogeranic acid ester by using esterification techniques known in the art (see in M. B. Smith, J. March, March's Advanced Organic Chemistry: Reactions, Mechanisms, and Structure. New York: Wiley, 2013). The obtained ester can be purified by distillation. Purified or crude homogeranic acid ester can be used for step B) as mix of its 3E/Z-Isomers in a chemical purity of >70%, most preferably >90%, most preferably in >95% purity. Trace compounds that can be present in the technically used homogeranic acid ester can be 2E/Z Homogeranic acid ester or also E/Z-Methyl-3-ethylidene-7-methyloct-6-enoate. The ratio of 3E:3Z-Isomers in homogeranic acid ester can vary.

In an embodiment, the step of esterification is carried out in the presence of sulfuric acid, NaHSO₄, KHSO₄, Amberlyst®, p-toluenesulfonic acid, methane sulfonic acid, formic acid or any other acidic catalyst. Preferably in the presence of sulfuric acid or NaHSO₄.

### Use:

In an embodiment the compounds prepared according to the process of the present invention can be used in the fragrance industry as intermediates or the compounds could be used as such as aroma compound.

In spite of a multitude of existing aroma chemicals (fragrances and flavorings) and processes for preparation thereof, there is a constant need for novel components in order to be able to satisfy the multitude of properties desired for the extremely diverse fields of use and for simple synthesis routes in order to make them available. The process of the invention enables the effective preparation of compounds of the general formula (I) that can serve as synthesis units of interest in the provision of novel aroma chemicals.

In a preferred embodiment, the compound of formula (I), may be used as aroma chemical in compositions selected from perfumes, detergents and cleaning compositions, cosmetic agents, body care agents, hygiene articles, products for oral and dental hygiene, scent dispensers, fragrances and pharmaceutical agents.

### EMBODIMENTS

In the following, there is provided a list of embodiments to further illustrate the present disclosure without intending to limit the disclosure to the specific embodiments listed below.
1. A process for preparing an ester compound of the general formula (I)

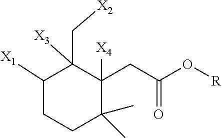

compound of formula (I)
   where,
   X₁ and X₃ together are the second bond of a double bond between the carbon atoms to which they are bound, with the proviso that X₂ and X₄ are hydrogen; or
   X₃ and X₄ together are the second bond of a double bond between the carbon atoms to which they are bound, with the proviso that X₁ and X₂ are hydrogen; or
   or its stereoisomers,
   wherein formula (I) comprises,
   the compound of formula (Ia)

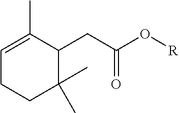

compound of formula (Ia)
   or its stereoisomers or mixture of its stereoisomers
   and the compound of formula (Ib)

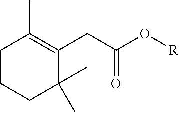

compound of formula (Ib)
   wherein R is selected from C₁-C₅ linear or branched alkyl and C₃-C₅ linear or branched alkenyl,
   comprising at least the step of:
   A) Providing a compound of formula (III)

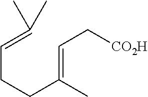

compound of formula (III)
   where R is selected from C₁-C₅ linear or branched alkyl and C₃-C₅ linear or branched alkenyl,
      or its stereoisomers, or mixture of its of stereoisomers,
   B) Cyclizing the compound of formula (III) to obtain compound of formula (I) in the presence of a catalyst selected from Brønsted acid or Lewis acid,
   C) Optionally purifying the compound of formula (I) obtained in step B).
2. The process according to embodiment 1, wherein R is selected from C₁-C₅ linear or branched alkyl and C₃-C₅ linear or branched alkenyl.
3. The process according to embodiment 1 or 2, wherein R is selected from methyl, ethyl, propyl, butyl, isobutyl, isopropyl, 1-propenyl, or 2-propenyl, preferably R is selected from methyl or ethyl.
4. The process according to embodiment 1, wherein in step B) the catalyst is selected from the group consisting of
   a) Lewis acids in the form of MAₓ where M is a metal, and A is a non-coordinating, weakly coordinating anion, alkoxide or a halogen and x is the valence of M. or
   b) Brønsted acid selected from the group consisting of mineral acids, mineral acid salts, organic acids, acid anhydrides (that can act as Brønsted acid precursors and can form free acids upon contact with a protic reagent), solid acid catalyst, or combinations thereof.
5. The process according to embodiment 4, wherein the step B) is carried out in the presence of an Brønsted acid selected from,
   mineral acids selected from hydrochloric acid, nitric acid, phosphoric acid, sulfuric acid, boric acid, hydrofluoric acid, hydrobromic acid, or hydroiodic acid, phosphonic acid, wherein the mineral acids immobilized on silica or any other thermostable support,
   mineral acid salts selected from potassium bisulfate, sodium bisulfate, Sodium dihydrogen phosphate,
   organic acids selected from p-toluenesulfonic acid, methanesulfonic acid, formic acid, acetic acid, oxalic acid, or trifluoroacetic acid,
   acid anhydrides that can act as Brønsted acid precursors and can form free acids upon contact with a protic reagent such as phosphorous pentoxide (P₂O₅), carbon dioxide (CO₂), Sulfur trioxide (SO₃), acetic anhydride (Ac₂O), methanesulfonic anhydride.
   solid acid catalysts selected from heteropoly acids, acid resin-type catalysts, strongly acidic ion exchangers, meso-porous silicas, acid clays, sulfated zirconia, molecular sieve materials, or acidic material on a thermo-stable support, or zeolites.
   or combinations thereof.
6. The process according to any of the embodiments 4 to 5, wherein the step B) is carried out in the presence of an Brønsted acid selected from phosphoric acid, p-toluene sulfonic acid, phosphonic acid or strongly acidic ion exchangers.
7. The process according to embodiment 6, wherein the Brønsted acid is phosphoric acid or trifluoracetic acid or silica supported phosphoric acid.
8. The process according to embodiment 7, wherein the Brønsted acid is phosphoric acid.
9. The process according to embodiment 4, wherein the step B) is carried out in the presence of a Lewis acid in the form of MAₓ where M is a metal, and A is a non-coordinating, weakly coordinating anion, alkoxide or a halogen and x is the valence of M wherein M comprises a transition metal, lanthanoid metal, or metals from Group 2, 3, 4, 5, 12, 13, 14 and 15 of the periodic table of the elements, and combinations thereof.
10. The process according to embodiment 9, wherein the metal M is selected from the group of elements iron, magnesium, zinc, boron, titanium, manganese, scandium, yttrium, lanthanum, europium, zirconium, aluminium, ytterbium, tin, vanadium, bismuth, scandium, or hafnium.
11. The process according to embodiment 9, wherein A is a non-coordinating, weakly coordinating anion selected from the group of a trifluoromethane sulfonate or triflate ([CF₃SO₃]⁻), hexafluorophosphate ([PF₆]⁻), [Al[OC(CF₃)₃]₄]⁻, tetrafluoroborate ([BF₄]⁻), perchlorate ([ClO₄]⁻), BArF ([B(ArHₓF_{y})₄]⁻ where Ar is an aryl and x+y=5), tosylate ([CH₃C₆H₄SO₃]⁻), mesylate ([CH₃SO₃]⁻), or antimony hexafluoride ([SbF₆]⁻).
12. The process according to embodiment 9, wherein A is a halogen selected from the group of chlorine, fluorine, iodine and bromine.
13. The process according any of the embodiments 9 to 12, wherein the Lewis acid is selected from scandium triflate [Sc(CF₃SO₃)₃], aluminium triflate [Al(CF₃SO₃)₃], hafnium triflate [Hf(CF₃SO₃)₄], yttrium triflate [Y(CF₃SO₃)₃], bismuth triflate [Bi(CF₃SO₃)₃] or ytterbium triflate [Yb(CF₃SO₃)₃], FeCl₃, FeBr₃, Me₂AlCl, TiCl₃(OiPr), AlCl₃, ZnCl₂, MgCl₂, BCl₃, SbCl₅ and its salts, SiCl₄, InCl₃ and its salts, GaCl₃, ZrCl₄, NbCl₅, TaCl₅, and its salts, Al(OTf)₃, BF₃, SnCl₄, or TiCl₄.
14. The process according any of the embodiments 9 to 13, wherein the Lewis acid is selected from FeCl₃, scandium triflate [Sc(CF₃SO₃)₃], aluminium triflate [Al(CF₃SO₃)₃], hafnium triflate [Hf(CF₃SO₃)₄], yttrium triflate [Y(CF₃SO₃)₃], bismuth triflate [Bi(CF₃SO₃)₃], or ytterbium triflate [Yb(CF₃SO₃)₃].
15. The process according to any one of the embodiments 1 to 14, wherein the catalyst in step B) is present in an amount in the range of 0.01 mol % to 100 mol % based on total amount of compound of formula (III).
16. The process according to any one of the embodiments 1 to 15, wherein the catalyst in the reaction is present in an amount in the range of 0.01 mol % to 50 mol % based on total amount of compound of formula (III).
17. The process according to any one of the embodiments 1 to 16, wherein the catalyst in the reaction is present in an amount in the range of 0.1 mol % to 10 mol % based on total amount of compound of formula (III).
18. The process according to any one of the embodiments 1 to 17, wherein the catalyst in the reaction is present in an amount in the range of 0.1 mol % to 5 mol % based on total amount of compound of formula (III).
19. The process according to any of the embodiments 1 to 18, wherein in step B), reaction is carried out at a temperature in the range of 0° C. to 150° C.
20. The process according to any of the embodiments 1 to 19, wherein in step B), reaction is carried out at a temperature in the range of 20° C. to 130° C.
21. The process according to any of the embodiments 1 to 20, wherein in step B), reaction is carried out at a temperature in the range of 50° C. to 120° C.
22. The process according to any of the embodiments 1 to 21, wherein in step B), reaction is carried out in the presence or absence of a solvent.
23. The process according to embodiment 22, wherein the solvent is selected from of the group consisting of ketones, esters, aromatic solvents, aliphatic solvents, cyclic ethers, alcohols, ethers and mixtures thereof.
24. The process according to embodiment 23, wherein the solvent is selected from toluene, benzene, benzyl alcohol, chlorobenzene, benzonitrile, xylene, trifluorotoluene, nitrobenzene, cyclohexane, or n-heptane, hexane, octane, tetrahydrofuran, 2-methyltetrahydrofuran, methyl-tert-butyl ether, 1-pentanol, 1-hexanol, methanol, 1-butanol, 1-propanol, 2-propanol, acetonitrile, water, dimethylformamide, tetrahydrofuran, toluene, ethyl acetate, dichloromethane, 1,1,1,3,3,3-hexafluoroisopropanol, dioxane or ethanol.
25. The process according to embodiment 24, wherein the solvent is selected from toluene, cyclohexane, n-heptane, ethanol or methanol.
26. The process according to embodiment 25, wherein the solvent is selected from toluene, cyclohexane or n-heptane.
27. The process according to any one of the embodiments 1 to 26, wherein in step B), the reaction is carried out as a batch reaction or as a continuous reaction in a continuous reactor setup.
28. The process according to any one of the embodiments 1 to 27, wherein in step B), the reaction is carried out as a batch reaction for a time period in the range of 10 minutes to 24 hours.
29. The process according to any one of the embodiments 1 to 27, wherein in step B), the reaction is carried out in a continuous reactor setup such as a mixing pump with a residence time in the range of 1 min to 10 hours.
30. The process according to any of the embodiments 1 to 29, wherein the compound of formula (I) includes a compound of formula (Ic)

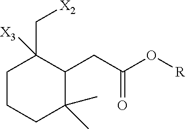

compound of formula (Ic)
   where,
   X₂ and X₃ together are the second bond of a double bond between the carbon atoms to which they are bound,
   R is selected from C₁-C₅ linear or branched alkyl and C₃-C₅ linear or branched alkenyl.
31. The process according to embodiment 30, wherein the compound of formula (Ic) is

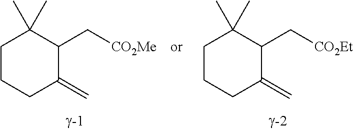

32. The process according to embodiment 1, wherein the compound of formula (III) is obtained by a process comprising at least the steps of:
   AA) providing a compound of formula (II)

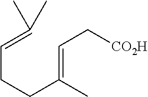

compound of formula (II)
   BB) Subjecting the compound of formula (II) to esterification reaction to obtain a compound of formula (III).
33. The process according to embodiment 32, wherein the step of esterification is carried out in the presence of sulfuric acid, KHSO₄, Amberlyst®, PTSA, MSA, formic acid or any other acidic catalyst.
34. The process according to embodiment 33, wherein the step of esterification is carried out in the presence of sulfuric acid.
35. The process according to any of the embodiments 1 to 29, wherein the compound of formula (I) includes compound of formula (V)

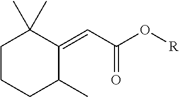

compound of formula (V)
   or its stereoisomers or mixture of its stereoisomers,
   where,
   R is selected from C₁-C₅ linear or branched alkyl and C₃-C₅ linear or branched alkenyl,
   in an amount less than 10 wt. %.

### Advantages of the Present Invention

1) The products are obtained in high yields.
2) The reaction conditions could be varied to obtain the α/β-Cyclohomogeranate in various ratios. The different ratio of α/β-Cyclohomogeranate in the final product can find use in several applications.
3) The process can be carried out as a batch process or as a continuous process.
4) Reaction time is relatively short.
5) The reaction does not involve expensive reagents.
6) The process avoids the use of waste producing reagents (no protecting group chemistry) and thereby reduces the number of purification steps.
7) The process leads to the formation of a lower amount of side product in the form of compound of formula (IV).

### EXAMPLES

Having described the invention, a further understanding can be obtained by reference to certain specific examples which are provided herein for the purposes of illustration only. The examples are not intended to be limiting unless otherwise specified.

### 1. Materials and Methods

### Materials

### Chemicals

Chemicals were purchased from commercial vendors (ABCR, Acros Organics, Alfa Aesar, Apollo Scientific, Fluorochem, Manchester Organics, Sigma-Aldrich, TCI) and used without further purification unless otherwise noted. Triethylamine, diisopropylamine and diisopropylethylamine were distilled over CaH₂ under argon atmosphere prior to use.

### Analytical Methods

### Thin-Layer Chromatography (TLC)

Monitoring reactions, analysis of column fractions and determination of retardation factors (R_{F} values) was performed by thin-layer chromatography on silica gel 60 (0.20 mm) with F₂₅₄ fluorescence indicator from Machery-Nagel. Qualitative analysis and visualization was accomplished by irradiation with UV light at λ=254 nm and/or by immersion in different staining reagents (specified for each compound in the respective experimental procedure) followed by heating with a heat-gun at 300° C. until dryness. The following stain was used (CAM stain proved to be particularly useful for the visualization of the cyclized lactone and ether products): Ce(SO₄)₂ (cerium sulfate: 5.0 g) and (NH₄)₆Mo₇O₂₄ 4H₂O (ammonium molybdate 25.0 g) were dissolved in H₂O (450 mL) and concentrated H₂SO₄ (50 mL)

### Nuclear Magnetic Resonance Spectroscopy (NMR)

The characterization is done by ¹³C NMR and ¹H NMR. The ¹³C NMR and ¹H NMR spectra were measured on a Bruker AV-500 spectrometer.

### (Flash) Column Chromatography

(Flash) Column chromatography was performed using silica gel (60 Å, 230-400 mesh, particle size: 43-63 μm) from Merck or using distilled technical grade solvents. The solvent mixtures and volume ratios (v/v) used as mobile phase for chromatography are specified in the corresponding experiment. Flash column chromatography was performed in glass columns by applying slightly elevated air or argon (0.3 mbar) pressure.

### Gas Chromatography (GC)

Gas Chromatography (GC) was performed on HP 6890 and 5890 Series instruments equipped with a split-mode capillary injection system and a flame ionization detector (FID) using hydrogen (H₂) as carrier gas. For quantitative GC-analysis of the reaction mixtures, the response factors of starting materials, identified intermediates, products and the internal standard were determined, and the quantification was validated using the calibration curve method for each respective component.

### 2. General Procedures

2.1 General Procedure A (Reactions at Room Temperature (r.t.) were Run Between 25 and 30° C. On a 0.20 mmol Scale)

The corresponding catalyst (the amount is specified in the table) was transferred to a 1.5 mL headspace screw-cap glass vial under ambient atmosphere and pressure and dissolved/suspended in the respective solvent (the concentration is indicated in the respective table). Methyl (E/Z)-homogeranate (43.5 μL, 39.3 mg, 0.20 mmol) and a PTFE-coated magnetic stir bar were added, the vial was closed with a screw-cap containing a PTFE/silicone septum and magnetically stirred (500 rpm) at r.t. (between 25 and 30° C.) for the indicated time. After the elapsed time, 1,3,5-trimethylbenzene (28 μL, 24.2 mg, 1.0 equiv.) was added as internal standard, the vial was shaken and stirred at r.t. for 5 min. An aliquot (typically between 5-10 μL) was removed, diluted with CDCl₃ (0.6 mL), filtered over solid anhydrous sodium carbonate and sodium sulfate. The conversion and yields of each individual component were subsequently analyzed by 1H NMR spectroscopy. Alternatively, the filtered solution was analyzed by GC spectroscopy.

### 2.2 General Procedure B (Reactions at T≥30° C. On a 0.20 Mmol Scale)

The corresponding catalyst (the respective amount is specified in the table) was transferred to a 2 mL headspace thick-walled crimp-cap glass vial under ambient atmosphere and pressure and dissolved/suspended in the specified solvent (the concentration is indicated in the respective table). Methyl (E/Z)-homogeranate (43.5 μL, 39.3 mg, 0.20 mmol) and a PTFE-coated magnetic stir bar were added, the vial was sealed with a crimp-cap containing a PTFE/silicone septum, placed inside a preheated aluminium block at the specified temperature and magnetically stirred (1000 rpm in the case of reactions of heterogeneous nature) at this temperature for the indicated time. After the elapsed time, the reaction mixture was allowed to cool to room temperature and 1,3,5-trimethylbenzene (28 μL, 24.2 mg, 1.0 equiv.) was added as internal standard. The vial was shaken and stirred at room temperature for 5 min. An aliquot (typically between 5-10 μL) was removed, diluted with CDCl₃ (0.6 mL), filtered over solid anhydrous sodium carbonate and sodium sulfate. The conversion and yields of each individual component were subsequently analyzed by ¹H NMR spectroscopy. Alternatively, the filtered solution was analyzed by GC spectroscopy.

Methyl homogeranate, that was used as starting material in the examples, was used with different isomeric compositions. As stated in the individual examples, either the pure Methyl (3E)-homogeranate was used as starting material or the isomeric mixture Methyl (E/Z)-homogeranate with a composition of 3E:3Z:2E=48:39:6.

3. Specific Examples: Synthesis of Methyl cyclohomogeranate (α-1) from Methyl (E/Z) Homogeranate

### 3.1 Evaluation of Brønsted Acids

Reactions were conducted on a 0.20 mmol scale according to general procedure B using the specified catalyst. Conversions and yields were determined by 1H NMR spectroscopy using CH₂Br₂ and/or 1,3,5-trimethylbenzene (preferably) as internal standard. Reactions with solid acid catalysts (Table 1, Exp. no. 1.4-1.9) were conducted as follows: DOWEX 50WX8 was acidified by treatment with H₂SO₄ (0.05 M) and subsequent washing with ethanol and dichloromethane followed by air drying prior to use. Montmorillonite K10, Amberlyst 15 and zeolites were commercially available and used as received. H₃PO₄ (20%, immobilized on silica) was available as beads and ground to a powder prior to use. The respective solid acid catalyst (10 mg, 50 mg/mmol loading) was transferred to a 2 mL headspace crimp-cap glass vial equipped with a PTFE-coated magnetic stir bar. Anhydrous toluene and the starting material (0.2 mmol) were added according to general procedure B, the vial was capped with a crimp-camp and placed inside a preheated aluminium heating block at 110° C. The resulting suspension was vigorously (1000 rpm) stirred for 2 h at this temperature. After the elapsed time, the mixture was diluted with MTBE (1 mL), filtered over NaHCO₃ and Na₂SO₄ (elution with 2×1 mL MTBE) and concentrated under reduced pressure to afford the crude product as a yellow oil. Conversion and isomer ratio of the crude product was determined by gas chromatography.

TABLE 1
Catalyst screening in the cyclization reaction of methyl homogeranate to methyl
cyclohomogeranate.

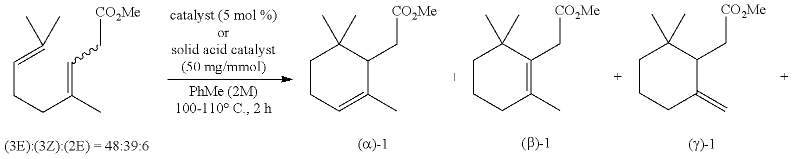

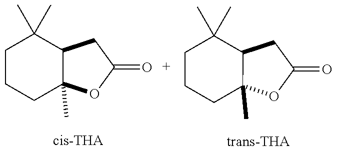

| | | Conversion/ | (α)-1/ | (β)-1/ | (γ)-1/ | cis-THA/ |
|---|---|---|---|---|---|---|
| Exp. no | catalyst (5 mol %) | % | % | % | % | % |
| 1.1 | H₃PO₄ (85% aqueous) | 96 | 41 | 37 | 4 | 14 |
| 1.2 | (+)-CSA | 11 | 5 | 3 | 1 | n.d. |
| 1.3 | p-TsOH•H₂O | 57 | 13 | 15 | 3 | n.d. |
| 1.4 | Amberlyst 15 | 92 | 12 | 19 | 1 | 22 |
| 1.5 | DOWEX 50WX8 | 99 | 6 | 10 | 4 | 39 |
| 1.6 | montmorillonite K10 | 97 | 22 | 39 | 3 | 25 |
| 1.7 | Zeolite Y (H form) | 69 | 25 | 20 | 5 | 2 |
| 1.8 | Zeolite Y (Na form) | 75 | 39 | 10 | 14 | 4 |
| 1.9 | H₃PO₄ (20%, | 97 | 26 | 44 | 2 | 15 |
| | immobolized on silica) | | | | | |

### 3.2 Evaluation of Phosphoric Acid as Brønsted Acid Catalyst

TABLE 2
Catalyst screening in the cyclization reaction of
methyl homogeranate to methyl cyclohomogeranate.

| Exp. | catalyst | Conversion/ | (α)-1/ | (β)-1/ | (γ)-1/ | cis-THA/ |
|---|---|---|---|---|---|---|
| no | (5 mol %) | % | % | % | % | % |
| 2.1 | H₃PO₂ (50% | 10 | 1 | 1 | 1 | n.d. |
| | aqueous) | | | | | |
| 2.2 | H₃PO₃ | 16 | 8 | 4 | 3 | n.d. |
| 2.3 | H₃PO₄ (85% | >95 | 42 | 29 | 6 | 10 |
| | aqueous) | | | | | |
| 2.4 | H₃PO₄ | >95 | 48 | 34 | 5 | 10 |
| | (crystalline) | | | | | |
| 2.5 | polyphosphoric | >95 | 47 | 45 | 3 | 5 |
| | acid | | | | | |

Reactions were conducted according to general procedure B at 110° C. using the specified catalyst. Conversions and yields were determined by 1H NMR spectroscopy using 1,3,5-trimethylbenzene as internal standard. The results are summarized in table 2.

3.2.1 Effect of Water on the Reaction with Phosphoric Acid as Brønsted Acid Catalyst

The effect of water on conversion, yield and selectivity in the cyclization reaction was investigated. A specific amount of water was added to the reaction mixture (the results are listed below in Table 3).

TABLE 3
Effect of the water content on the outcome of the phosphoric acid-catalyzed cyclization
reaction of methyl homogeranate to methyl cyclohomogeranate.

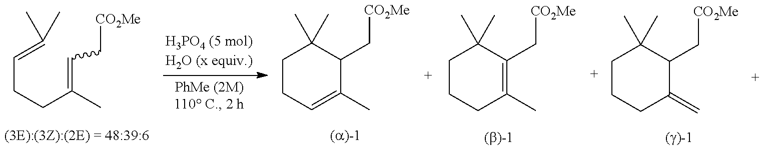

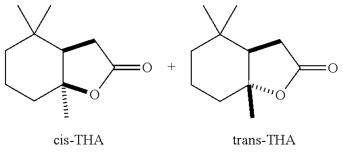

| | | conversion/ | (α)-1/ | (β)-1/ | (γ)-1/ | cis-THA/ |
|---|---|---|---|---|---|---|
| Exp. no | H₂O (equiv.) | % | % | % | % | % |
| 3.1 | 0 | >95 | 48 | 36 | 4 | 8 |
| 3.2 | 0.2 | 73 | 30 | 16 | 7 | 15 |
| 3.3 | 0.5 | 64 | 19 | 11 | 7 | 14 |
| 3.4 | 1 | 52 | 16 | 10 | 6 | 12 |
| 3.5 | 2 | 40 | 11 | 8 | 4 | 8 |

Reactions were conducted according to general procedure B at 110° C. using crystalline phosphoric acid as catalyst. The amount added water is specified in each entry. Conversions and yields were determined by 1H NMR spectroscopy using 1,3,5-trimethylbenzene as internal standard.

The results corroborate the previous findings (see Table 2) that anhydrous phosphoric acid sources consistently provided higher yields and selectivity in the cyclization reaction compared to aqueous phosphoric acid. A gradual increase of the water content led to a gradual decrease of the conversions and yields (see Table 3, examples 3.2-3.5).

### 3.3 Evaluation of Lewis Acids

Various Lewis acids were tested as catalysts in the cyclization reaction of methyl homogeranate to methyl cyclohomogeranate (table 4)

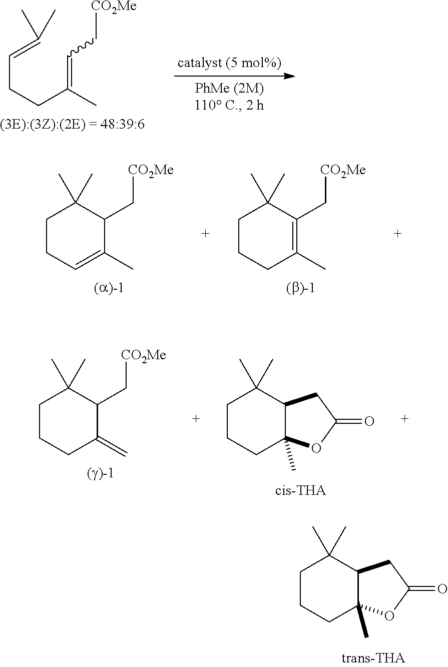

TABLE 4
Evaluation of Lewis acid catalysts for the cyclization reaction
of methyl homogeranate to methyl cyclohomogeranate. For comparison,
the results obtained with crystalline and 85% aqueous phosphoric
acid are provided in entries 4.8 and 4.9.

| Exp. | catalyst | conversion/ | (α)-1/ | (β)-1/ | (γ)-1/ | cis-THA/ |
|---|---|---|---|---|---|---|
| no | (5 mol %) | % | % | % | % | % |
| 4.1 | FeCl₃ | >95 | 32 | 51 | 2 | 13 |
| | (anhydrous) | | | | | |
| 4.2 | Al(OTf)₃ | >95 | 24 | 36 | 1 | 35 |
| 4.3 | Sc(OTf)₃ | >95 | 31 | 49 | 2 | 14 |
| 4.4 | Y(OTf)₃ | >95 | 32 | 56 | 2 | 5 |
| 4.5 | Yb(OTf)₃ · | >95 | 26 | 43 | 2 | 26 |
| | H₂O | | | | | |
| 4.6 | Bi(OTf)₃ | >95 | 13 | 19 | 1 | 65 |
| 4.7 | Hf(OTf)₄ · | >95 | 25 | 37 | 1 | 36 |
| | H₂O | | | | | |
| 4.8 | H₃PO₄ | >95 | 48 | 36 | 4 | 8 |
| | (crystalline) | | | | | |
| 4.9 | H₃PO₄ (85% | 83 | 39 | 24 | 7 | 13 |
| | aqueous) | | | | | |

Reactions were conducted according to general procedure B at 110° C. using the specified catalyst. Conversions and yields were determined by gas chromatography and/or ¹H NMR spectroscopy using 1,3,5-trimethylbenzene as internal standard.

Out of the tested catalysts, anhydrous FeCl₃, Sc(OTf)₃ and Y(OTf)₃ proved to be the most selective towards methyl cyclohomogeranate (>80%), while providing <15% of the cis-tetrahydroactinidiolide side product. Notably, in contrast to reactions with crystalline or 85% aqueous phosphoric acid, where the a-isomer is typically obtained as the major product, the Lewis acid-catalyzed process appears to provide the B-isomer as the major product (up to 56% in the case of yttrium triflate).

### 3.4 Variation of Reaction Conditions

### 3.4.1 Selection of Solvent

The cyclization reaction can be conducted in various solvents, preferably aliphatic or aromatic hydrocarbon solvents such as toluene, cyclohexane and n-heptane with comparable results regarding yield and selectivity towards methyl cyclohomogeranate. The ratio between the isomers and the cis-lactone side product slightly varies depending on the solvent (table 4).

TABLE 4
Optimization of the reaction solvent in the cyclization reaction
of methyl homogeranate to methyl cyclohomogeranate.

| Exp. | | conversion/ | (α)-1/ | (β)-1/ | (γ)-1/ | cis-THA/ |
|---|---|---|---|---|---|---|
| no | solvent | % | % | % | % | % |
| 5.1 | toluene | 97 | 40 | 48 | 2 | 7 |
| 5.2 | cyclohexane | 97 | 45 | 36 | 4 | 10 |
| 5.3 | n-heptane | 97 | 44 | 40 | 3 | 10 |

Reactions were conducted on a 0.20 mmol scale in the specified solvent according to general procedure B using crystalline phosphoric acid (5 mol %) as catalyst. Conversions and yields were determined by 1H NMR spectroscopy using 1,3,5-trimethylbenzene as internal standard.

### 3.4.2 Selection of the Concentration of the Starting Material.

The cyclization reaction can be conducted in a concentration range from 0.5 to 10 M, and concentrations ranging from 2 to 10 M were found to be optimal with respect to conversion of the methyl homogeranate starting material (see Table 5). Formation of the cis-THA side product was gradually suppressed with increasing dilution (≤2 M).

TABLE 5
Variation of the concentration in the synthesis of methyl
cyclohomogeranate (α-1) from methyl (E/Z)-homogeranate.

| Exp. | concentration/ | conversion/ | (α)-1/ | (β)-1/ | (γ)-1/ | cis-THA/ |
|---|---|---|---|---|---|---|
| no | M | % | % | % | % | % |
| 6.1 | 0.5 | 61 | 32 | 20 | 7 | n.d. |
| 6.2 | 1 | 85 | 44 | 30 | 6 | 5 |
| 6.3 | 2 | 97 | 39 | 44 | 3 | 8 |
| 6.4 | 4 | 98 | 45 | 32 | 4 | 11 |
| 6.5 | 5 | 98 | 42 | 34 | 4 | 13 |
| 6.6 | 10 | 98 | 39 | 40 | 3 | 13 |

Reactions were conducted on a 0.20 mmol scale in toluene (2 M) according to general procedure B using crystalline phosphoric acid (5 mol %) as catalyst. Conversions and yields were determined by ¹H NMR spectroscopy using 1,3,5-trimethylbenzene as internal standard. n.d.: not detected by ¹H NMR analysis.

### 3.4.3 Variation of the Catalyst Loading

The catalyst loading of crystalline phosphoric acid was varied between 2.5 to 100 mol %, preferably between 5 and 10 mol %, in order to ensure complete consumption of the starting material (within 2 h reaction time) and to minimize the amount of the cis-THA side product (Table 6).

TABLE 6
Variation of the catalyst loading in the synthesis of methyl
cyclohomogeranate (α-1) from methyl (E/Z)-homogeranate

| | catalyst | | | | | |
|---|---|---|---|---|---|---|
| Exp. | loading/ | conversion/ | (α)-1/ | (β)-1/ | (γ)-1/ | cis-THA/ |
| No | mol % | % | % | % | % | % |
| 7.1 | 100 | 99 | 15 | 24 | 1 | 42 |
| 7.2 | 50 | 99 | 21 | 34 | 1 | 36 |
| 7.3 | 25 | 99 | 27 | 41 | 3 | 26 |
| 7.4 | 10 | 97 | 39 | 41 | 3 | 14 |
| 7.5 | 5 | 97 | 39 | 44 | 3 | 11 |
| 7.6 | 2.5 | 79 | 40 | 30 | 9 | n.d. |

Reactions were conducted on a 0.20 mmol scale in toluene (2 M) according to general procedure B using crystalline phosphoric acid as catalyst. Conversions and yields were determined by ¹H NMR spectroscopy using 1,3,5-trimethylbenzene as internal standard. n.d.: not detected by ¹H NMR analysis.

### 3.4.4 Effect of Temperature

TABLE 7
Variation of the temperature in the synthesis of methyl
cyclohomogeranate (α-1) from methyl (E/Z)-homogeranate.

| Exp. | T/ | Conversion/ | (α)-1/ | (β)-1/ | (γ)-1/ | cis-THA/ |
|---|---|---|---|---|---|---|
| no | ° C. | % | % | % | % | % |
| 8.1 | 80 | 22 | 8 | 5 | 3 | n.d. |
| 8.2 | 100 | >95 | 40 | 48 | 2 | 7 |
| 8.3 | 110 | >95 | 48 | 34 | 5 | 10 |

Reactions were conducted on a 0.20 mmol scale in toluene (2 M) according to general procedure B using crystalline phosphoric acid as catalyst at the indicated temperature. Conversions and yields were determined by ¹H NMR spectroscopy using 1,3,5-trimethylbenzene as internal standard. n.d.: not detected by ¹H NMR analysis.

### Example 4: Cyclization of Isopropyl (3E)-Homogeranate

The effect of the isomeric purity on the outcome of the cyclization reaction was evaluated wherein isomerically pure isopropyl and methyl (3E)-homogeranate were used as the starting material.

The reactivity of the pure (3E)-isomers in the phosphoric acid-catalyzed cyclization reaction was investigated (Table 8: Phosphoric acid-catalyzed cyclization of isomerically pure methyl or isopropyl (3E)-homogeranate.). The screening revealed that the methyl ester was more reactive than the isopropyl ester. In addition, the amount of the (β)-1 was significantly increased compared to the reaction with the isomeric mixture (α)-1/(β)-1≈1:1.2 compared to 1:1.7 for the pure isomer).

TABLE 8
Phosphoric acid-catalyzed cyclization of isomerically pure methyl or isopropyl (3E)-
homogeranate.

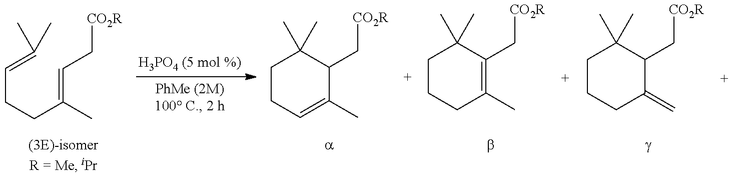

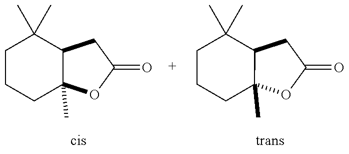

| Exp. No | R = | conversion/% | α/% | β/% | γ/% | cis-THA/% |
|---|---|---|---|---|---|---|
| 9.1 | Me | >95 | 28 | 48 | 2 | 11 |
| 9.2 | ⁱPr | 76 | 32 | 25 | 9 | 10 |

Reactions were conducted on a 0.20 mmol scale in toluene (2 M) according to general procedure B using crystalline phosphoric acid as catalyst at the indicated temperature. Conversions and yields were determined by 1H NMR spectroscopy using 1,3,5-trimethylbenzene as internal standard. n.d.: not detected by 1H NMR analysis.

### Example 5: Preparation of Alpha-Cyclohomogeranic Acid Methylester (α-1), Beta-Cyclohomogeranic Acid Methylester (β-1), Gamma-Cyclohomogeranic Acid Methylester (γ-1)

A flame-dried 4 mL screw-cap glass vial under argon was charged with (E/Z)-homogeranic acid methyl ester (mixture of 3 E:3Z:2E of 48:39:6, 392 mg, 2.00 mmol, 1.00 equiv) and a PTFE-coated magnetic stir bar. The starting material was dissolved in dry toluene (0.60 mL, 3.33 M), the vial was placed inside a preheated aluminum heating block and the resulting clear colorless solution was heated to 100° C. Then, H₃PO₄ (85% w/w solution in H₂O, 11.5 mg, 0.10 mmol, 0.05 equiv.) was added to the stirred reaction mixture, the pierced screw-cap was quickly replaced with a new one and the mixture was stirred (500 rpm) at 100° C. for 2 h (a gradual color change from a colorless solution with pink droplets of H₃PO₄ to a yellow solution with brown droplets within 15 min reaction time was observed). After the elapsed time, the yellow reaction mixture was allowed to cool to r.t., a small aliquot was analyzed by ¹H NMR spectroscopy and TLC indicating full consumption of the starting material and formation of the desired products (except for the conjugated 2E-isomer that does not appear to react under these reaction conditions). Aqueous saturated Na₂CO₃ solution was carefully added (gas evolution), the mixture was diluted with MTBE (2 mL), the organic phase was removed and the aqueous phase was extracted with MTBE (5×2 mL). The combined organic layers were dried over Na₂SO₄, filtered and concentrated under reduced pressure to afford the crude product as a yellow oil. Purification by flash column chromatography on silica gel (Merck, 40-63 μm, 230-400 mesh, 30 g) using hexanes/MTBE as eluent afforded the product as a mixture of isomers as a pale yellow oil (315 mg, 1.61 mmol, 80% yield, 84% GC-purity, the mixture contains approximately 6% of the 2E-isomer of methyl homogeranate).

| | TLC (hexanes/MTBE 19:1) | | | | | corrected |
|---|---|---|---|---|---|---|
| fraction | CAM stain (blue spots) | tara/g | weight/g | yield/mg | yield/% | yield/% |
| 8-13 | R_{f} = 0.31 and 0.37 | 4.5909 | 4.9061 | 315.2 | 80 | 67 |
| | (two spots) | | | | | |

A second reaction was set up in parallel on the same scale using crystalline H₃PO₄ (>99.9999%, 9.80 mg, 0.10 mmol, 0.05 equiv.). In this case, H₃PO₄ was weighed directly into the vial, suspended in toluene followed by addition of methyl homogeranate. Otherwise, procedure, observations, extraction and purification steps were essentially identical to those described above. Purification by flash column chromatography afforded the cyclized products in three fractions as a colorless or pale yellow oil (318 mg, 1.62 mmol, 81% yield, corrected yields based on the GC-purity are shown in the table below.)

| | TLC (hexanes/MTBE 19:1) | | | | | corrected |
|---|---|---|---|---|---|---|
| fraction | CAM stain | tara/g | weight/g | yield/mg | yield/% | yield/% |
| 4 | R_{f} = 0.37 (stains less | 4.6711 | 4.6768 | 5.70 | 1.5 | 1.4 |
| | intense, light blue spot) | | | | | |
| 5-7 | R_{f} = 0.31 and 0.37 | 4.6744 | 4.9699 | 295.5 | 75 | 64 |
| | (two spots) | | | | | |
| 8 | mainly R_{f} = 0.31 (slightly | 4.6663 | 4.6826 | 16.3 | 4.2 | 3.3 |
| | darker blue spot) | | | | | |

The analytical data/characterization for the compounds isolated from the reactions described in example 5 are provided below.

rac beta-cyclohomogeranic acid methylester (α-1)

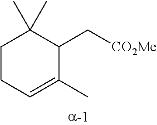

Analytical data for α-1, isolated as pure compound (>95% purity according to ¹H NMR and GC analysis) in fraction 4 of the column chromatography of the second reaction of example 5.

Physical appearance: colorless oil

TLC (SiO₂, hexanes/MTBE 19:1) Rₜ=0.37 (CAM stain, light blue spot)

¹H NMR (CD₂Cl₂, 501 MHZ) δ (ppm) 5.38-5.32 (m, 1H), 3.64 (s, 3H), 2.35 (dd, J=17.3, 8.1 Hz, 1H), 2.27-2.14 (m, 2H), 2.02-1.91 (m, 2H), 1.70-1.59 (m, 3H), 1.41-1.34 (m, 1H), 1.21-1.14 (m, 1H), 0.91 (s, 3H), 0.82 (s, 3H).

¹³C NMR (CD₂Cl₂, 126 MHZ) δ (ppm) 175.0, 135.6, 121.7, 51.8, 46.1, 36.0, 32.5, 31.7, 27.1, 26.3, 23.3, 22.8.

GC DB-Waxetr 0.25 mm/0.25 μm, 30 m, temperature: 220° C. (injector)/from 60° C. to 130° C. with 2° C./min, then with 12° C./min to 260° C., 350° C. (detector), gas: 0.60 bar H₂, sample size: 0.2 μL, t_{R}=25.39 min.

HRMS (GC-CI, ammonia) (m/z) calculated for C₁₂H₂₁O₂⁺ [M+H]⁺, 197.1536; found, 197.1537.

beta-cyclohomogeranic acid methylester (β-1)

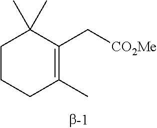

Analytical data for β-1, obtained as the major component in a mixture of isomers (according to ¹H NMR and GC analysis) in fraction 8 of the column chromatography of the second reaction of example 5.

Physical appearance: pale yellow oil

TLC (SiO₂, hexanes/MTBE 19:1) Rₜ=0.31 (CAM stain, light blue spot)

¹H NMR (CDCl3, 501 MHZ) δ (ppm)=3.66 (s, 3H), 3.05 (s, 2H), 2.01-1.96 (m, 2H), 1.65-1.54 (m, 2H), 1.58 (s, 3H), 1.48-1.44 (m, 2H), 0.96 (s, 6H).

¹³C NMR (CDCl3, 126 MHz) δ (ppm)=173.5, 131.6, 130.5, 51.8, 39.5, 34.9, 33.7, 32.8, 28.1, 20.4, 19.5.

GC DB-Waxetr 0.25 mm/0.25 mm, 30 m, temperature: 220° C. (injector)/from 60° C. to 135° C. with 2° C./min, then with 6° C./min to 220° C., then with 12° C./min to 260° C., then 5 min at 260° C., 350° C. (detector), gas: 0.60 bar H₂, sample size: 0.2 μL, t_{R}=29.0 min (GC-MS: m/z [M]+=196).

HRMS (GC-EI) m/z calculated for C₁₂H₂₀O₂+ [M]⁺: 196.1458; found: 196.1457.

rac-gamma-cyclohomogeranic Acid Methylester (γ-1)

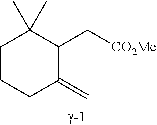

The γ-isomer was identified as minor compound in fraction 5-7 and 8 of the second reaction of example 5 and isolated.

TLC (SiO₂, hexanes/MTBE 19:1) Rₜ=0.31 (CAM stain, light blue spot)

¹H NMR (CDCl₃, 501 MHZ) δ (ppm)=4.75 (q, J=1.4 Hz, 1H), 4.56 (s, 1H), 3.64 (s, 3H), 2.50 (t, J=10.2 Hz, 1H), 2.46-2.38 (m, 2H), 2.22 (dt, J=12.7, 6.1 Hz, 1H), 2.05 (ddd, J=13.1, 8.2, 5.3 Hz, 1H), 1.43 (ddd, J=11.6, 7.2, 4.3 Hz, 1H), 1.35 (ddd, J=13.3, 8.1, 4.9 Hz, 1H), 0.96 (s, 3H), 0.79 (s, 3H).

GC DB-Waxetr 0.25 mm/0.25 mm, 30 m, temperature: 220° C. (injector)/from 60° C. to 135° C. with 2° C./min, then with 6° C./min to 220° C., then with 12° C./min to 260° C., then 5 min at 260° C., 350° C. (detector), gas: 0.60 bar H₂, sample size: 0.2 μL, t_{R}=29.71 min (99%).

HRMS (GC-CI, ammonia) (m/z) calculated for C₁₂H₂₁O₂⁺ [M+H]⁺: 197.1536; found, 197.1537.

### Example 6: Preparation of Alpha-Cyclohomogeranic Acid Methylester (α-1), Beta-Cyclohomogeranic Acid Methylester (β-1), Gamma-Cyclohomogeranic Acid Methylester (γ-1)

A 100 mL round-bottom flask was charged with (E/Z)-methyl homogeranate (3.92 g, 20 mmol, 1.0 equiv.) and a PTFE-coated magnetic stir bar. The starting material was dissolved in hexafluoroisopropanol (20 mL, 9.5 equiv., 1 M) and TFA (trifluoroacetic acid, 2.38 g, 20.9 mmol, 1.04 equiv.) was added dropwise (an immediate color change from colorless over bright yellow then bright orange to dark orange-red was observed upon addition of TFA within 1 min) to the stirred solution and the resulting red solution was stirred at 25° C. for 24 h. After the elapsed time, the dark brown-red reaction mixture was concentrated under reduced pressure. The residue was dissolved in hexanes/MTBE (19:1 v/v, 20 mL), Celite was added and the slurry was concentrated under reduced pressure. Purification by flash column chromatography on silica gel (Merck, 40-63 μm, 300 g) using hexanes/MTBE as eluent followed by drying in vacuo overnight afforded Methyl cyclohomogeranate as a colorless oil (2.34 g, 11.9 mmol, 60% yield, with α-1:β-1: γ-1=31:68:1 based on GC).

1.-19. (canceled)

20. A process for preparing an ester compound of the general formula (I) 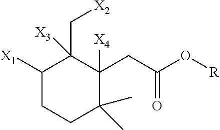
compound of formula (I)
where,
X₁ and X₃ together are the second bond of a double bond between the carbon atoms to which they are bound, with the proviso that X₂ and X₄ are hydrogen; or
X₃ and X₄ together are the second bond of a double bond between the carbon atoms to which they are bound, with the proviso that X₁ and X₂ are hydrogen; or
or its stereoisomers,
wherein formula (I) comprises,
the compound of formula (Ia) 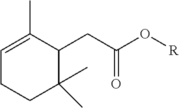
compound of formula (Ia)
or its stereoisomers or mixture of its stereoisomers, and
the compound of formula (Ib) 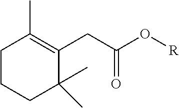
compound of formula (Ib)
wherein R is selected from C₁-C₅ linear or branched alkyl and C₃-C₅ linear or branched alkenyl, comprising at least the step of:
A) providing a compound of formula (III) 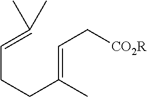
compound of formula (III)
or its stereoisomers, or mixture of its of stereoisomers,
B) cyclizing the compound of formula (III) to obtain compound of formula (I) in the presence of a catalyst selected from Brønsted acid or Lewis acid,
C) optionally purifying the compound of formula (I) obtained in step B).

21. The process according to claim 20, wherein R is selected from methyl, ethyl, propyl, butyl, isobutyl, isopropyl, 1-propenyl, or 2-propenyl.

22. The process according to claim 20, wherein in step B) the catalyst is selected from the group consisting of,
a) Lewis acids in the form of MAₓ where M is a metal, and A is a non-coordinating, weakly coordinating anion, alkoxide or a halogen and x is the valence of M,
or
b) Brønsted acid selected from the group consisting of mineral acids, mineral acid salts, organic acids, solid acid catalyst, or combinations thereof.

23. The process according to claim 22, wherein the step B) is carried out in the presence of an Brønsted acid selected from,
mineral acids selected from hydrochloric acid, nitric acid, phosphoric acid, sulfuric acid, boric acid, hydrofluoric acid, hydrobromic acid, or hydroiodic acid, phosphonic acid, wherein the mineral acids immobilized on silica or any other thermostable support,
mineral acid salts selected from potassium bisulfate, sodium bisulfate, Sodium dihydrogen phosphate,
organic acids selected from p-toluenesulfonic acid, methanesulfonic acid, formic acid, acetic acid, oxalic acid, or trifluoroacetic acid,
solid acid catalysts selected from heteropoly acids, acid resin-type catalysts, strongly acidic ion exchangers, meso-porous silicas, acid clays, sulfated zirconia, molecular sieve materials, or acidic material on a thermo-stable support, or zeolites,
or combinations thereof.

24. The process according to claim 22, wherein step B) is carried out in the presence of Brønsted acid selected from phosphoric acid, p-toluene sulfonic acid, phosphonic acid or strongly acidic ion exchangers.

25. The process according to claim 24, wherein the Brønsted acid is phosphoric acid, trifluoracetic acid, or silica supported phosphoric acid.

26. The process according to claim 22, wherein the step B) is carried out in the presence of a Lewis acid in the form of MAₓ where M is a metal, and A is a non-coordinating, weakly coordinating anion, alkoxide, or a halogen and x is the valence of M wherein M comprises a transition metal, lanthanoid metal, or metals from Group 2, 3, 4, 5, 7, 8, 12, 13, 14 and 15 of the periodic table of the elements, and combinations thereof.

27. The process according to claim 26, wherein the metal M is selected from the group of elements iron, magnesium, zinc, boron, titanium, scandium, yttrium, lanthanum, europium, zirconium, manganese, aluminium, ytterbium, tin, vanadium, bismuth, scandium, or hafnium.

28. The process according to claim 26, wherein A is a non-coordinating, weakly coordinating anion selected from trifluoromethane sulfonate or triflate ([CF₃SO₃]₋), hexafluorophosphate ([PF₆]₋), [Al[OC(CF₃)₃]₄]⁻, tetrafluoroborate ([BF₄]), perchlorate ([ClO₄]⁻), BArF ([B(ArHₓF_{y})4]⁻ where Ar is an aryl and x+y=5), tosylate ([CH₃C₆H₄SO₃]⁻), mesylate ([CH₃SO₃]⁻), or antimony hexafluoride ([SbF₆]⁻).

29. The process according to claim 26, wherein A is a halogen selected from the group of chlorine, fluorine, iodine and bromine.

30. The process according to claim 26, wherein the Lewis acid is selected from scandium triflate [Sc(CF₃SO₃)₃], aluminium triflate [Al (CF₃SO₃)₃], hafnium triflate [Hf(CF₃SO₃)₄], yttrium triflate [Y(CF₃SO₃)₃], bismuth triflate [Bi(CF₃SO₃)₃] or ytterbium triflate [Yb(CF₃SO₃)₃], FeCls, FeBr₃, MnCl₂, BiCl₃, Me₂AlCl, TiCl₃(OiPr), AlCl₃, ZnCl₂, ZnBr₂, Zn(OTf)₂, MgCl₂, BCl₃, Al(OTf)₃, BF₃, SnCl₄, or TiCl₄.

31. The process according to claim 20, wherein the catalyst in the reaction is present in an amount in the range of 0.01 to 100 mol % based on the total amount of compound of formula (III).

32. The process according to claim 20, wherein in step B), reaction is carried out at a temperature in the range of 0 to 150° C.

33. The process according to claim 20, wherein in step B), reaction is carried out in the presence or absence of a solvent.

34. The process according to claim 33, wherein the solvent is selected from of the group consisting of ketones, esters, aromatic solvents, aliphatic solvents, cyclic ethers, alcohols, water, nitriles, ethers and mixtures thereof.

35. The process according to claim 34, wherein the solvent is selected from toluene, benzene, benzyl alcohol, chlorobenzene, benzonitrile, xylene, trifluorotoluene, nitrobenzene, cyclohexane, or n-heptane, hexane, octane, tetrahydrofuran, 1-pentanol, 1-hexanol, methanol, 1-butanol, 1-propanol, 2-propanol, tetrahydrofuran, 2-methyl tetrahydrofuran, methyl tert-butyl ether, toluene, ethyl acetate, acetonitrile, water, dimethylformamide, dichloromethane, 1,1,1,3,3,3-hexafluoroisopropanol, dioxane or ethanol.

36. The process according to claim 20, wherein in step B), the reaction is carried out as a batch reaction or in a continuous reactor setup.

37. The process according to claim 20, wherein the compound of formula (I) further includes a compound of formula (Ic) 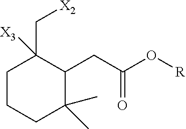
compound of formula (Ic)
where,
X₂ and X₃ together are the second bond of a double bond between the carbon atoms to which they are bound,
R is selected from C₁-C₅ linear or branched alkyl and C₃-C₅ linear or branched alkenyl.

38. The process according to claim 20, wherein the compound of formula (I) includes compound of formula (V) 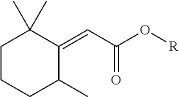
compound of formula (V)
or its stereoisomers or mixture of its stereoisomers,
where,
R is selected from C₁-C₅ linear or branched alkyl and C₃-C₅ linear or branched alkenyl, in an amount less than 10 wt. %.
